(19) United States
(12) Patent Application Publication
(10) Pub. No.: US 2010/0087392 A1
(43) Pub. Date: Apr. 8, 2010

(54) COMPLEXES OF IBUPROFEN, CYCLODEXTRINS AND TERNARY AGENTS AND THE USE THEREOF IN PHARMACEUTICS

(75) Inventors: Bernard Freiss, Castres (FR); Hubert Lochard, Brens (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Pierre Fabre Medicament, Boulogne (FR)

(21) Appl. No.: 12/450,391

(22) PCT Filed: Mar. 26, 2008
(86) PCT No.: PCT/FR2008/000416

§ 371 Date: Sep. 23, 2009

(30) Foreign Application Priority Data
Mar. 28, 2007 (FR) 0702246

(51) Int. Cl. A61K 31/724; A61P 29/00

(52) U.S. Cl. 514/58

(57) ABSTRACT

The present invention relates to pharmaceutical compositions comprising ibuprofen in the form of an ibuprofen/cyclodextrin/lysine complex having improved pharmacokinetic properties compared with ibuprofen alone, such as a shortened Tmax and an increased Cmax.

The present invention relates to complexes of ibuprofen, cyclodextrin and ternary agent (arginine or lysine), such complexes having a substantially increased solubility and being capable of being obtained by the technology of dense fluids under pressure, especially CO₂.

The present invention relates also to the use of those complexes in the pharmaceutical field.

Numerous active substances, especially of interest in the pharmaceutical field, exhibit very low solubility or are insoluble in water and consequently in biological liquids. This implies low bioavailability of those active substances and a substantial increase in the doses administered to patients to achieve the set therapeutic objective, with the consequent increase in the possible side effects associated with medical treatments.

In the particular case of anti-inflammatory active ingredients, it is also important that the pharmacological action be as rapid as possible and that the exposure to the said active ingredient also be as high as possible.

The present invention relates to a pharmaceutical composition containing ibuprofen in the form of an ibuprofen/cyclodextrin/ternary agent complex.

In the context of the present invention, the ternary agent is arginine or lysine. Advantageously, the said complex is capable of being obtained by a complexing process comprising a step for diffusing the ibuprofen/cyclodextrin/arginine or ibuprofen/cyclodextrin/lysine mixture in the presence of a dense fluid under pressure and at least one diffusing agent.

The present invention relates also to a pharmaceutical composition containing ibuprofen in the form of an ibuprofen/cyclodextrin/arginine or ibuprofen/cyclo-dextrin/lysine complex that is capable of being obtained by a complexing process comprising a step for diffusing the ibuprofen/cyclodextrin/arginine or ibuprofen/cyclodextrin/lysine mixture in the presence of a dense fluid under pressure and at least one diffusing agent, which composition is characterised in that, after oral administration to the rat, the absorption of ibuprofen is such that:
the Cmax is 10 to 20 times higher
and/or the AUC is 7 to 10 times greater
than the Cmax and/or AUC obtained with a pharmaceutical form containing ibuprofen alone administered to the same animal at the same dose.

Advantageously, the absorption of ibuprofen is such that the Tmax is from 1.5 to 2.5 times lower than the Tmax obtained with a pharmaceutical form containing ibuprofen alone administered to the same animal at the same dose.

In the context of the present invention, “cyclodextrins” are understood to mean cyclodextrins, modified cyclodextrins and mixtures thereof. The cyclodextrin is advantageously β-cyclodextrin, methyl-β-cyclodextrin, γ-cyclodextrin or hydroxypropyl-β-cyclodextrin. Advantageously, it is β-cyclodextrin.

In the context of the present invention, “dense fluid under pressure” is understood to mean any fluid used at a temperature or pressure higher than its critical value. The fluid is advantageously pure CO₂ or CO₂ mixed with an organic solvent conventionally used by the person skilled in the art.

In the context of the present invention, “diffusing agent” is understood to mean any solvent that promotes an interaction of ibuprofen and cyclodextrins.

Advantageously, the diffusing agent is selected from the group composed of alcohols, ketones, ethers, esters and water with or without surfactant and mixtures thereof. Even more advantageously, the diffusing agent is water.

The parameters relating to the absorption and to the metabolism of ibuprofen in the rat that are used to characterise the invention are as follows:
Cmax corresponds to the maximum plasma concentration of ibuprofen over time. It is expressed in ng/ml.

Tmax, expressed in hours, is the time required to reach Cmax
AUC is the area under the curve “plasma concentration as a function of time” from T0 (oral administration of ibuprofen) to T8 (8 hours post administration). It is expressed in ng.h/ml.

The composition according to the present invention allows a far more intense and rapid absorption of the active ingredient and thus a distinct improvement in exposure to ibuprofen compared with the compositions of the prior art.

Ibuprofen is more rapidly and more amply absorbed with greater maximum plasma concentrations in corresponding shorter times.

Advantageously, the present invention relates to a pharmaceutical composition containing ibuprofen in the form of an ibuprofen/cyclodextrin/arginine or ibuprofen/cyclodextrin/lysine complex that is capable of being obtained by a complexing process comprising a step for diffusing the ibuprofen/cyclodextrin/arginine or ibuprofen/cyclodextrin/lysine mixture in the presence of a dense fluid under pressure and at least one diffusing agent, which composition is characterised in that, after oral administration to the rat, the absorption of ibuprofen is such that:
the Cmax is from 12.5 to 18 times higher
and/or the AUC is from 7.5 to 9 times greater
than the Cmax and/or AUC obtained with a pharmaceutical form containing ibuprofen alone administered to the same animal at the same dose.

Preferably, the absorption of ibuprofen is such that the Tmax is 2 times lower than the Tmax obtained with a pharmaceutical form containing ibuprofen alone administered to the same animal at the same dose.

It will be possible for the active substance/cyclodextrin/ternary agent molar ratio to be so selected as to ensure optimum inclusion of the active substance in the cyclodextrin.

Advantageously, the ibuprofen/cyclodextrin/ternary agent (arginine or lysine) complexes according to the present invention are such that between 0.1 mol and 3 mol inclusive, preferably between 0.5 mol and 2 mol inclusive, of cyclodextrin, and between 0.1 mole and 3 mol inclusive, preferably between 0.5 mol and 2 mol inclusive, of arginine or lysine, are present per mol of ibuprofen. Accordingly, the pharmaceutical composition according to the present invention is preferably characterised in that the ibuprofen/cyclodextrin molar ratio is between 1/0.1 and 1/3 inclusive, and the ibuprofen/lysine molar ratio is between 1/0.1 and 1/3 inclusive. Similarly, the pharmaceutical composition according to the present invention is preferably characterised in that the ibuprofen/cyclodextrin molar ratio is between 1/0.1 and 1/3 inclusive and the ibuprofen/arginine ratio is between 1/0.1 and 1/3 inclusive.

Preferably, the complex according to the present invention is characterised by a molar ratio of 1/1/1 of ibuprofen, cyclodextrin and ternary agent (arginine or lysine) respectively.

The ibuprofen, cyclodextrin and ternary agent (arginine or lysine) complexes according to the present invention are capable of being obtained by a process such as that described hereinbelow.

A process for the preparation of a complex according to the present invention comprises the following successive steps:
a) placing ibuprofen in contact with a cyclodextrin and the ternary agent (arginine or lysine),
b) carrying out a molecular diffusion step by placing in contact, in static mode, a dense fluid under pressure and the mixture obtained in step (a) in the presence of one or more diffusing agents,
c) recovering the ibuprofen-cyclodextrin-ternary agent (arginine or lysine) complex so formed.

Step (b), molecular diffusion in static mode, termed the maturation step, consists essentially of a molecular diffusion phase in dense medium under pressure, especially supercritical medium, which allows the inclusion of ibuprofen in the cyclodextrins. The desired objective during that diffusion phase is the formation of inclusion complexes between ibuprofen, cyclodextrin and the ternary agent (arginine or lysine).

The complex so formed associates, in a non-covalent manner, ibuprofen, the cyclodextrin and the ternary agent (arginine or lysine).

The ternary agent (arginine or lysine), which is used as interaction agent, interacts in accordance with two plausible hypotheses: strong interaction with the ibuprofen included in the cyclodextrin and/or strong interaction with the complex formed.

The presence of the ternary agent (arginine or lysine) allows principally the dissolution properties of the complex in biological liquids, especially water, to be improved, and the level of inclusion of ibuprofen in the cyclodextrin to be increased.

The improvement in the physico-chemical properties, especially in terms of dissolution and bioavailability of the system formed, may be as a result of
a non-covalent interaction of the ternary agent (arginine or lysine) with the ibuprofen, the cyclodextrin or both (complex formation, salt formation . . . )
a local variation of the pH of the dissolution medium
obtaining a system having a eutectic
a modification of the interface between the system and its dissolution medium (surfactant effect, granulometry change).

In the context of the present invention “static mode” is understood to mean a reaction or a process in which all of the reagents are simultaneously brought together and the reaction is allowed to proceed. For example, in step (b) of the present invention, ibuprofen, water, the ternary agent (arginine or lysine) and supercritical CO₂ are introduced into an autoclave and left to react for several hours.

The mass of material does not change during the reaction. Conversely, in dynamic mode, the reagents are added as required by the development of the reaction or of the production. In a dynamic-mode arrangement, a fluid is often circulated. The mass of material changes during the production.

Advantageously, the molecular diffusion step (b) of the process according to the present invention is carried out with stirring.

In a particular embodiment of the invention, during step (a) ibuprofen, ternary agent (arginine or lysine) and cyclodextrins are introduced in solid or liquid form into a receptacle into which dense fluid under pressure and the diffusing agent are injected in judiciously selected proportions. The pressure and temperature conditions and also the duration of the treatment are determined by any appropriate method.

The diffusing agent may be added continuously or discontinuously in an amount of between 1 and 50% by weight relative to the total weight of the mixture, advantageously between 20 and 25% by weight relative to the total weight of the mixture.

The time required for the molecular diffusion of step (b) is determined by any appropriate method. Step (b) can be repeated as many times as desired to obtain a satisfactory dissolution rate. Advantageously, step (b) lasts for between approximately 1 and 16 hours, advantageously 2 hours.

The pressure and temperature conditions of step (b) are so selected as to promote molecular diffusion. Advantageously, the pressure of the supercritical fluid is between 5 MPa and 40 MPa inclusive and is advantageously 15 MPa, and the temperature is between 0 and 120° C. inclusive and is advantageously 100° C.

Advantageously, step (b) of the process according to the present invention is carried, out in a closed reaction vessel, especially an autoclave.

The process may be carried out batchwise or continuously. The process according to the present invention is advantageously carried out batchwise.

Carrying out the molecular diffusion step in dense medium under pressure in the presence of a diffusing agent allows a strong interaction between the ibuprofen particles and the cyclodextrins, which promotes dissolution in aqueous medium.

The pharmacokinetic characteristics of the complex according to the present invention have been demonstrated in the rat. Those characteristics allow the applicant to consider the use of those complexes in the field of the preparation of a human medicament intended for analgesic or anti-inflammatory use.

The pharmacokinetic and bioavailability characteristics of the ibuprofen contained in a complex according to the present invention have been demonstrated in man. The results obtained are disclosed in Example 5 hereinbelow.

From that clinical study it can be observed that the ratios relating to the pharmacokinetic parameters Cmax and Tmax are, as a result of inter-species variability, different from those observed with the rat but are no less significant of a substantially increased therapeutic efficacy of the complex according to the invention compared with ibuprofen administered on its own.

Accordingly, in man it may be noted that, for an orally administered amount of ibuprofen of 200 mg, the Tmax of ibuprofen alone is 50% greater than the Tmax observed with the ibuprofen of the complex according to the invention. As to the Cmax, it appears that, for the complex according to the invention, again at a dose of 200 mg, this parameter represents 136% of the Cmax observed for ibuprofen alone. Finally, it is of particular interest to note that for a dose of ibuprofen in the form of the complex according to the present invention that is two times lower (100 mg instead of 200 mg), and in relation to ibuprofen alone, the Tmax is still shortened to the same extent, and the Cmax is reduced only by less than 20%.

This makes it possible to consider using the complex according to the invention with lower doses of ibuprofen compared with conventional commercial compositions containing ibuprofen alone, while at the same time obtaining the same antalgic and/or anti-inflammatory effects, or even substantially improved effects, and/or limiting side effects.

The present invention thus relates also to the use of a composition according to the present invention for the preparation of a medicament having an anti-inflammatory effect at a dose of ibuprofen per administration of between 50 mg and 400 mg inclusive.

One of the objects of the present invention is the said anti-inflammatory pharmaceutical composition for oral administration which is characterised in that the dose of ibuprofen per administration is between 50 mg and 400 mg inclusive. Preferably, an oral administration amount of ibuprofen in complex form according to the present invention of between approximately 100 mg and approximately 300 mg inclusive, and even more preferably of approximately 200 mg, will be used for the preparation of a medicament having an anti-inflammatory effect.

An important point of the present invention is the direct impact of the plasma profile (Cmax, AUC, Tmax) of ibuprofen obtained with a composition according to the invention on the dose to be administered.

Given the better absorption of the active ingredient and the greater exposure thereto at the same dose of ibuprofen, the composition according to the invention will have a greater therapeutic effect than a composition of the prior art.

Thus, the administration of a composition according to the invention at a dose conventionally used for an antalgic effect, that is to say between 50 and 400 mg/administration and preferably approximately 200 mg/administration, allows an anti-inflammatory activity to be obtained in the case of the present invention (most often 600 mg per administration is required when using a composition of the prior art).

In the same way, with a composition according to the invention a smaller dose of ibuprofen will be used than with a composition of the prior art to achieve an identical therapeutic effect.

Finally, the applicant has demonstrated that the maximum plasma concentrations of ibuprofen in the rat after oral administration of a composition according to the present invention are:
on the one hand greater (by a factor of 10 to 20)
and on the other hand these maximum concentrations are achieved in shorter post-administration times
   than the plasma concentrations obtained with a pharmaceutical form containing ibuprofen alone administered to the same animal at the same dose.

The Cmax can reasonably be expected to be reached in approximately 15 minutes in this animal.

These characteristics illustrate the rapid and intense absorption of ibuprofen. This “flash” effect of the product administered at an equivalent dose is especially advantageous in human therapeutics when the desire is to obtain an antalgic or anti-inflammatory effect very quickly after oral administration in order to ensure almost immediate relief from pain.

### Evaluation of the Complexation Level of Ibuprofen

The complexation level of the active substance in the substrate is evaluated by differential thermal analysis (DSC).

In DSC, a temperature gradient is applied under nitrogen flux to the product being tested.

The complexation yield is evaluated by measuring the reduction (or disappearance) of the thermal peak relative to the melting of the active ingredient “remaining free” in crystalline form.

### Ibuprofen Solubilisation Test

### Procedure Protocol:

The amount of ibuprofen in the dissolution solution is determined by HPLC:

### Apparatus Employed:

### WATERS HPLC System:

separation module 2695
UV detector 2487

### Chromatographic Conditions:

Column: NUCLEOSIL C18 150×4.6 mm.

### Mobile Phase:

Channel A: acetonitrile

Channel B: 0.5 ml phosphoric acid, 340 ml acetonitrile

Water ad 1000 ml.

### Elution Gradient:

| Time (min) | % A | % B |
|---|---|---|
| 0 | 25 | 75 |
| 10 | 25 | 75 |
| 15 | 70 | 30 |
| 20 | 70 | 30 |
| 25 | 25 | 75 |
| 30 | 25 | 75 |

Flow rate: 2 ml/min
Detector wavelength: 214 nm
Detector sensitivity: 2 AUFS
Volume injected: 20 μl
Oven temperature: 30° C.
Analysis time: 30 minutes

### Preparation of the Control Solutions:

SM: Introduce 200 mg of the control ibuprofen into a 100 ml flask. Dissolve with 50 ml of acetonitrile and make up the volume with water.
Range:
   T1: Dilution to 1/40 of SM in 50 acetonitrile/50 water.
   T2: Dilution to 1/20 of T3 in 50 acetonitrile/50 water.
   T3: Dilution to 1/10 of SM in 50 acetonitrile/50 water.
   T4: Dilution to 3/20 of SM in 50 acetonitrile/50 water.
   T5: Dilution to 1/5 of SM in 50 acetonitrile/50 water.

### Operating Conditions of the Dissolution Kinetics at 2 g/l:

Phosphate Buffer Preparation pH 3.0 (Ph. Eur. 4000500):

Dilute 0.7 ml of phosphoric acid R in 100 ml of water R. Supplement to 900 ml with the same solvent and adjust to pH 3.0 with concentrated sodium hydroxide solution R then supplement to 1000 ml with water R.

### Test Procedure:

### Operating Conditions:

Introduce into a 100 ml Erlenmeyer flask a test administration amount equivalent to 100 mg of ibuprofen. Add 50 ml of buffer pH 3.0. Subject to magnetic stirring at 400 revolutions per minute in a bath thermostatically maintained at 37° C.±2° C. Remove a 2 ml sample, with magnetic stirring, at 15, 30, 60, 120 minutes. Filter the samples through Gelman GHP Acrodisc 0.45 μm polypropylene filter. The solution must be clear.

### Methodology, Expression of the Results:

Inject 20 μl of each control solution.

Carry out a linear regression of the areas of the ibuprofen peaks relative to the concentrations. The correlation coefficient must be greater than 0.995.
Inject 20 μl of the solutions to be examined.

Measure the area of the ibuprofen peak in each solution being examined. Deduce therefrom the concentration X in μg/ml according to the regression line of the controls.

The level of solubilisation of ibuprofen indicated in the example is the average of the solubilisation levels over 15, 30, 60 and 120 minutes calculated by dividing the concentration of solubilised ibuprofen by the total ibuprofen concentration in the starting solution.

The following Examples, given for information only, were carried out with ibuprofen, β-cyclodextrin, and water as diffusing agent.

### EXAMPLE 1

### Ibuprofen, β-cyclodextrin Complexes

2 grams of ibuprofen, 12.6 grams of β-cyclodextrin are introduced into a reaction vessel, as well as 1.7 grams of diffusing agent (water). Carbon dioxide is subsequently introduced into the reaction vessel under a pressure of 15 MPa and at a temperature of 60° C. The whole is maintained under those operating conditions for a period of two hours.

The solubilisation level and the complexation level are measured for the complexes obtained.

The ibuprofen solubilisation level is close to 90%.

The complexation level is evaluated by DSC as 96.6%.

### EXAMPLE 2

### Ibuprofen, β-cyclodextrin, Arginine Complexes

2 grams of ibuprofen, 12.6 grams of β-cyclodextrin and 1.7 grams of arginine are introduced into a reaction vessel, as well as 2.1 grams of diffusing agent (water). Carbon dioxide is subsequently introduced into the reaction vessel under a pressure of 15 MPa and at a temperature of 60° C. The whole is maintained under those operating conditions for a period of two hours.

The complexation level and the solubilisation level are measured for the complexes obtained.

The ibuprofen solubilisation level is 100%.

The complexation level is evaluated by DSC as 100%.

### EXAMPLE 3

### Ibuprofen, β-cyclodextrin and L-Lysine Complexes

2 grams of ibuprofen, 12.6 grams of β-cyclodextrin and 1.4 grams of lysine are introduced into a reaction vessel, as well as 2.1 grams of diffusing agent (water). Carbon dioxide is subsequently introduced into the reaction vessel under a pressure of 15 MPa and at a temperature of 60° C. The whole is maintained under those operating conditions for a period of two hours.

The complexation level and the solubilisation level are measured for the complexes obtained.

The ibuprofen solubilisation level is 100%.

The complexation level is evaluated by DSC as 100%.

### EXAMPLE 4

### Pharmacokinetic Evaluation

An exploratory comparative pharmacokinetics study was carried out in the rat for different ibuprofen complexes administered in a single dose by the oral route.

### Study Synopsis:

Dose administered=20 mg/kg ibuprofen (IBU)
6 groups of n=3 animals per group
The different compositions administered are as follows:
   IBU alone
   IBU+arginine (molar ratio 1/1)
   IBU+lysine (molar ratio 1/1)
   IBU+arginine+β-cyclodextrin (according to Example 2)
   IBU+lysine+β-cyclodextrin (according to Example 3)
   IBU+β-cyclodextrin (according to Example 1)
The carrier used for the preparation of these compositions is distilled water.
Blood sampling times: blood samples are taken at T0 (on administration) then at 0.25-0.5-1-2-4-6-8 and 24 hours post administration
Measurement of the plasma concentrations of ibuprofen: The plasma samples are precipitated in acetonitrile prior to analysis by HPLC-MS-MS.
Apparatus used: TSQ quantum with “Selected Reaction Monitoring”
   detection mode: negative ion mode
   capillary temperature: 325° C.
   capillary voltage: 4.0 kV
   The transition of the molecular ion 2205.2 m/z is followed towards the fragment 161.2 m/z

Chromatographic Conditions:
Column: Synergi Fusion-RP 80, 2×50 mm, 4 microns

Mobile Phase:
Channel A: 13.3 mM ammonium formate/6.7 mM formic acid in water
Channel B: 6 mM ammonium formate/3 mM formic acid in water/acetonitrile (1/9, v/v)

Elution Gradient:

| | | | Flow rate |
|---|---|---|---|
| Time (min) | % A | % B | (ml/min) |
| 0 | 60 | 40 | 0.5 |
| 1 | 60 | 40 | |
| 2.5 | 0 | 100 | |
| 3.5 | 0 | 100 | |
| 3.6 | 60 | 40 | |
| 4.5 | 60 | 40 | |

Plot of the curve/obtaining the plasma profile=plasma concentration as a function of the time post administration

Calculation of the Pharmacokinetic Parameters:
The parameters below are obtained using KINETICA® software known to the person skilled in the art
   Cmax expressed in ng/ml corresponds to the observed maximum plasma concentration of ibuprofen
   Tmax expressed in hours is the time post administration to reach Cmax
   AUC or AUCT8 expressed in ng.h/m1 corresponds to the area under the curve up to 8 hours post administration. The concentrations obtained at the last sampling time, 24 hours, are lower than the quantification limit.

### Results Obtained:

The results obtained are presented in the table below:

| | | | | IBU + | IBU + | |
|---|---|---|---|---|---|---|
| | | IBU + | IBU + | Arg + | Lys + | |
| | IBU alone | Arg | Lys | β-cycl | β-cycl | IBU + β-cycl |
| Cmax | 2276 | 12003 | 12089 | 29599 | 41813 | 11744 |
| (ng/ml) | | | | | | |
| Tmax (h) | 0.5 | 0.5 | 0.5 | 0.25 | 0.25 | 1 |
| AUC | 7188 | 37491 | 53251 | 55062 | 63314 | 29064 |
| (ng · h/ml) | | | | | | |
| Cmax/ | 1.00 | 5.27 | 5.31 | 13.00 | 18.37 | 5.16 |
| Cmax_{IBUalone} | | | | | | |
| AUC/ | 1.00 | 5.22 | 7.41 | 7.66 | 8.81 | 4.04 |
| AUC_{IBUalone} | | | | | | |

### CONCLUSIONS

Compared with ibuprofen alone, the greatest increases in plasma ibuprofen exposures are observed in the presence of ibuprofen/cyclodextrin/arginine or ibuprofen/cyclodextrin/lysine complexes.

Indeed, the maximum concentrations Cmax of ibuprofen achieved with the cyclodextrin/arginine or cyclodextrin/lysine complexes are higher than those obtained with the other forms tested.

Finally, the corresponding Tmax values are likewise lower, which demonstrates a more rapid absorption of ibuprofen using the compositions according to the invention.

In the case of the ibuprofen/cyclodextrin/arginine complex, in the rat the absorption of ibuprofen administered by the oral route is such that:
Tmax is 2 times lower
Cmax is 13 times higher
AUC is 7.7 times greater
than the Tmax, Cmax, AUC obtained with the pharmaceutical form containing ibuprofen alone administered to the same sample of animals at the same dose.

In the case of the ibuprofen/cyclodextrin/lysine complex, in the rat the absorption of ibuprofen administered by the oral route is such that:
Tmax is 2 times lower
Cmax is 18.4 times higher
AUC is 8.8 times greater
than the Tmax, Cmax, AUC obtained with the pharmaceutical form containing ibuprofen alone administered to the same sample of animals at the same dose.

The addition of cyclodextrin with arginine or lysine, and preferably with lysine (complex prepared by supercritical fluid technology) thus substantially improves intestinal absorption in the rat by the oral route.

### EXAMPLE 5

### Clinical Study

A clinical study was conducted to evaluate the pharmacokinetic parameters and bioavailability after the oral administration to 24 healthy volunteers of a single dose of 3 forms of ibuprofen:
ibuprofen/cyclodextrin/lysine complex according to the invention containing 100 mg of ibuprofen (according to Example 3)
ibuprofen/cyclodextrin/lysine complex according to the invention containing 200 mg of ibuprofen (according to Example 3)
ibuprofen alone, 200 mg

### Methodology

The volunteers are randomly divided into three groups and receive a single oral administration of one of the specialties tested (ibuprofen/cyclodextrin/lysine, 200 mg of ibuprofen; ibuprofen/cyclodextrin/lysine, 100 mg of ibuprofen; ibuprofen 200 mg).

Blood samples were taken to determine the plasma concentration of ibuprofen at T0 and 15 min., 20 min., 25 min., 30 min., 45 min., 1 h., 1 h 15, 1 h 30, 1 h 45, 2 h, 3 h, 4 h, 6 h, 10 h, 14 h and 24 h after administration.

This procedure, administration/sample-taking/dosage, was repeated seven times over a period of seven weeks at a rate of once per week.

The results represent the average of the 7 tests and are presented in the table below:

| | | IBU + Lys + | IBU + Lys + |
|---|---|---|---|
| | IBU alone | β-cycl | β-cycl |
| | 200 mg | 100 mg IBU | 200 mg IBU |
| Cmax | 11 | 9 | 15 |
| (μg/ml) | | | |
| Tmax (min) | 90 | 60 | 60 |
| Cmax/ | — | 82% | 136% |
| Cmax_{IBUalone} | | | |
| Change in | — | −33% | −33% |
| Tmax | | | |

It may be noted that, at an identical amount of active ingredient, that is to say 200 mg of ibuprofen, the use of the complex according to the invention allows a Cmax representing 136% of the Cmax of the active ingredient alone to be obtained and, referring to the Tmax, a 33% curtailment of that parameter is noted at that same dose.

In respect of the complex according to the invention containing 100 mg of ibuprofen, it may be noted that, compared with double the dose of ibuprofen administered alone, the Tmax is still reduced by 33%. As regards the Cmax, it is notable to observe that, in the case of the complex according to the invention, the Cmax still represents 82% of the value of the Cmax of double the non-complexed dose.

1-20. (canceled)

21. A pharmaceutical composition comprising ibuprofen in the form of an ibuprofen/cyclo-dextrin/lysine complex, wherein the complex is obtained by a complexing process comprising a step of diffusing an ibuprofen/cyclodextrin/lysine mixture and at least one diffusing agent in the presence of a dense fluid under pressure.

22. The pharmaceutical composition of claim 21, wherein the dense fluid under pressure is carbon dioxide.

23. The pharmaceutical composition of claim 21, wherein the diffusing agent is selected from alcohols, ketones, ethers, esters, water, water with surfactant, and mixtures thereof.

24. The pharmaceutical composition of claim 21, wherein the ibuprofen/cyclodextrin molar ratio is between 1/0.1 and 1/3 inclusive and the ibuprofen/lysine molar ratio is between 1/0.1 and 1/3 inclusive.

25. The pharmaceutical composition of claim 24, wherein the ibuprofen/cyclodextrin molar ratio is 1/1 and the ibuprofen/lysine molar ratio is 1/1.

26. The pharmaceutical composition of claim 21, wherein the absorption of ibuprofen after oral administration to a rat exhibits,
a Cmax 10 to 20 times higher, and/or
a AUC 7 to 10 times greater,
than the Cmax and/or AUC exhibited with a pharmaceutical form containing ibuprofen alone administered to the same animal at the same dose.

27. The pharmaceutical composition of claim 26, wherein the Tmax is from 1.5 to 2.5 times lower than the Tmax exhibited with a pharmaceutical form containing ibuprofen alone administered to the same animal at the same dose.

28. The pharmaceutical composition of claim 21, which is to be administered orally, and wherein the amount of ibuprofen is between 50 mg and 400 mg inclusive.

29. A method for anti-inflammatory treatment comprising the step of administering to a human or animal subject, an effective amount of the pharmaceutical composition of claim 21.

30. The method of claim 29, wherein the dose of ibuprofen per administration is 200 mg.

31. A pharmaceutical composition comprising ibuprofen in the form of an ibuprofen/cyclodextrin/arginine complex, wherein the complex is obtained by a complexing process comprising a step of diffusing an ibuprofen/cyclodextrin/arginine mixture and at least one diffusing agent in the presence of a dense fluid under pressure.

32. The pharmaceutical composition of claim 31, wherein the dense fluid under pressure is carbon dioxide.

33. The pharmaceutical composition of claim 31, wherein the diffusing agent is selected from alcohols, ketones, ethers, esters, water, water with surfactant, and mixtures thereof.

34. The pharmaceutical composition of claim 31, wherein the ibuprofen/cyclodextrin molar ratio is between 1/0.1 and 1/3 inclusive and the ibuprofen/arginine molar ratio is between 1/0.1 and 1/3 inclusive.

35. The pharmaceutical composition of claim 34, wherein the ibuprofen/cyclodextrin molar ratio is 1/1 and the ibuprofen/arginine molar ratio is 1/1.

36. The pharmaceutical composition of claim 31, wherein the absorption of ibuprofen after oral administration to the rat exhibits,
a Cmax 10 to 20 times higher, and/or
a AUC 7 to 10 times greater,
than the Cmax and/or AUC exhibited with a pharmaceutical form containing ibuprofen alone administered to the same animal at the same dose.

37. The pharmaceutical composition of claim 36, wherein the Tmax is from 1.5 to 2.5 times lower than the Tmax exhibited with a pharmaceutical form containing ibuprofen alone administered to the same animal at the same dose.

38. The pharmaceutical composition of claim 31, which is to be administered orally, and wherein the amount of ibuprofen is between 50 mg and 400 mg inclusive.

39. A method for anti-inflammatory treatment comprising the step of administering to a human or animal subject, an effective amount of the pharmaceutical composition of claim 31.

40. The method of claim 39, wherein the dose of ibuprofen per administration is 200 mg.
